(19) United States
(12) Patent Application Publication
(10) Pub. No.: US 2025/0325776 A1
(43) Pub. Date: Oct. 23, 2025

(54) VALVE ASSEMBLY FOR OXYGEN DELIVERY DEVICE AND METHODS OF USE

(71) Applicant: Jason Semple, St. George, UT (US)

(72) Inventor: Jason Semple, St. George, UT (US)

(21) Appl. No.: 19/176,882

(22) Filed: Apr. 11, 2025

Related U.S. Application Data
(60) Provisional application No. 63/635,262, filed on Apr. 17, 2024.

(51) Int. Cl. A61M 16/20; A61M 16/00; A61M 16/08

(52) U.S. Cl. CPC A61M 16/201; A61M 16/0078; A61M 16/0816; A61M 16/208; A61M 2202/0208

(57) ABSTRACT

An oxygen delivery device includes an oxygen inlet, a switching valve, an oxygen reservoir, a self-inflating bag, an oxygen outlet tube, and an oxygen outlet from the self-inflating bag. When the switching valve is in a first position, oxygen is diverted to the oxygen outlet tube where it is provided to a spontaneously breathing patient. When the switching valve is in a second position, oxygen flows into the reservoir bag and the self-inflating bag, where a clinician may pump the bag to assist the breathing of non-spontaneously breathing patient. In some versions, a clamp may be used in place of the switching valve.

### CROSS-REFERENCE TO RELATED APPLICATIONS

This application claims the benefit of U.S. Provisional Application Ser. No. 63/635,262, filed on Apr. 17, 2024, which is incorporated herein by reference.

### TECHNICAL FIELD

The present disclosure relates to oxygen delivery masks/devices. In particular, the present disclosure relates to a self-inflating resuscitation bag having a valve configured to allow spontaneous breathing.

### BACKGROUND

Intubation is a medical procedure where a tube is inserted into a patient's airway (trachea) to help them breathe. This tube can be placed through the mouth or nose and is often connected to a machine that delivers air or oxygen. Intubation is typically performed in emergencies or before surgeries when a person cannot breathe on their own due to conditions like airway obstruction, respiratory failure, or cardiac arrest. It can be a life-saving intervention, ensuring that the airway remains open and air can reach the lungs.

In the moments preceding intubation, patients are often still breathing spontaneously, meaning they are breathing independently, or with minimal assistance. During this period, clinicians utilize a free-flowing oxygen delivery device. However, they must also have a self-inflating resuscitation bag (which does not provide free-flowing oxygen and should not be used to provide oxygen to a spontaneously breathing patient) readily available for immediate use when the patient ceases spontaneously breathing. This scenario presents several challenges for the clinician.

Firstly, there is a limited availability of oxygen sources to operate the necessary oxygen delivery devices. In the given example, a clinician would require three oxygen sources, at minimum: one for the device providing free-flowing oxygen to the spontaneously breathing patient, a second for the self-inflating resuscitation bag to be used when the patient stops breathing spontaneously, and a third for the ventilator to be employed once the intubation procedure is completed and the patient is stabilized. However, in many cases, a clinician may only have access to two oxygen sources in a patient's room.

If three oxygen sources are not available, the clinician must disconnect one device once it is no longer needed and then connect another device that is required. This process necessitates the clinician diverting their attention and focus away from the patient, who is in a critical condition and undergoing a high-risk procedure. Often, the oxygen source that needs to be accessed is beyond arm's reach, requiring the clinician to physically step away from the patient to switch devices at the oxygen source. This action introduces unnecessary risk.

Accordingly, there is a need for an oxygen mask/device that is capable of delivering free-flowing oxygen to a spontaneously breathing patient, while further providing a self-inflating resuscitation bag once the patient is not spontaneously breathing. The present disclosure seeks to solve these and other problems.

### SUMMARY OF EXAMPLE EMBODIMENTS

In some embodiments, an oxygen delivery device comprises an oxygen inlet, a switching valve, an oxygen reservoir, a self-inflating bag, an oxygen outlet tube, and an oxygen outlet from the self-inflating bag. When the switching valve is in a first position, oxygen is diverted to the oxygen outlet tube where it is provided to a spontaneously breathing patient. When the switching valve is in a second position, oxygen flows into the reservoir bag and the self-inflating bag, where a clinician may squeeze the bag to assist the breathing of a non-spontaneously breathing patient.

In some embodiments, a switching valve may have three openings, allowing a clinician to supply oxygen through an oxygen outlet tube when in a first position, through a reservoir port in a second position, and through both the oxygen outlet tube and the reservoir port simultaneously when in a third position.

In some methods of use, a clinician will couple the oxygen delivery device with switching valve in a first position to a spontaneously breathing patient. Once the patient ceases spontaneously breathing, the clinician may actuate the switching valve to a second position, directing oxygen into the reservoir bag and self-inflating bag, where the clinician may then squeeze the bag to assist the breathing of the non-spontaneously breathing patient. In some methods, a clinician may actuate the valve to a third position, allowing oxygen to flow to the patient in both directions, allowing a patient to inhale free-flowing oxygen as well as access oxygen within the reservoir.

In some embodiments, an oxygen delivery device comprises an oxygen inlet, a three-way port, an oxygen reservoir, a self-inflating bag, an oxygen outlet tube, a clamp positioned on the oxygen outlet tube, and an oxygen outlet from the self-inflating bag. When the clamp is open, oxygen may freely flow through the oxygen outlet tube where it is provided to a spontaneously breathing patient. When the clamp is closed, oxygen flows into the reservoir bag and the self-inflating bag, where a clinician may squeeze the bag to assist the breathing of a non-spontaneously breathing patient.

### BRIEF DESCRIPTION OF THE DRAWINGS

FIG. 1 illustrates a side perspective view of an oxygen delivery mask with a switching valve in a first position;

FIG. 2 illustrates a side perspective view of an oxygen delivery mask with a switching valve in a second position;

FIG. 3 illustrates an enlarged view of a switching valve in a first position of an oxygen delivery mask;

FIG. 4 illustrates an enlarged view of a switching valve in a second position of an oxygen delivery mask;

FIG. 5 illustrates an enlarged view of a switching valve in a first position of an oxygen delivery mask;

FIG. 6 illustrates an enlarged view of a switching valve in a second position of an oxygen delivery mask;

FIG. 7 illustrates an enlarged view of a switching valve in a third position of an oxygen delivery mask; and

FIG. 8 illustrates a side perspective view of an oxygen delivery mask with a clamp.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As previously discussed, there is a need for an oxygen delivery device that is capable of delivering free-flowing oxygen to a spontaneously breathing patient, while further allowing for a self-inflating resuscitation bag once the patient is not spontaneously breathing. The oxygen delivery device disclosed herein solves problems related to patient safety, clinician attentiveness, and timeliness of intubation procedures, as well as other problems.

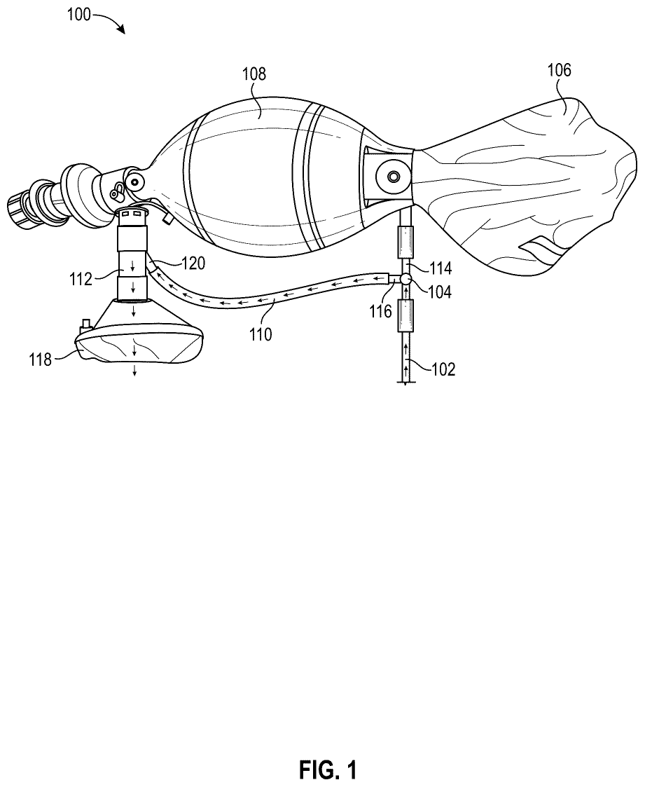

Referring to FIG. 1, in some embodiments, an oxygen delivery device 100 comprises an oxygen inlet 102, a switching valve 104, a reservoir bag 106, a self-inflating bag 108, an oxygen outlet tube 110, and a patient port 112 from the self-inflating bag 108. When the switching valve 104 is in a first position, the reservoir port 114 is closed and the outlet tube port 116 is open, thereby diverting oxygen (flow of oxygen illustrated by arrows) into the oxygen outlet tube 110 and to the patient port 112, where it is provided to a spontaneously breathing patient. As a result, a patient who is still spontaneously breathing (e.g., not sedated), can continue to breathe normally. Further, the patient may also be properly preoxygenated, also known as hyperoxygenated, prior to the procedure, which may aid in extending the safe apnea time (i.e., the duration of time following cessation of breathing/ventilation until critical arterial desaturation occurs) and avoiding hypoxia.

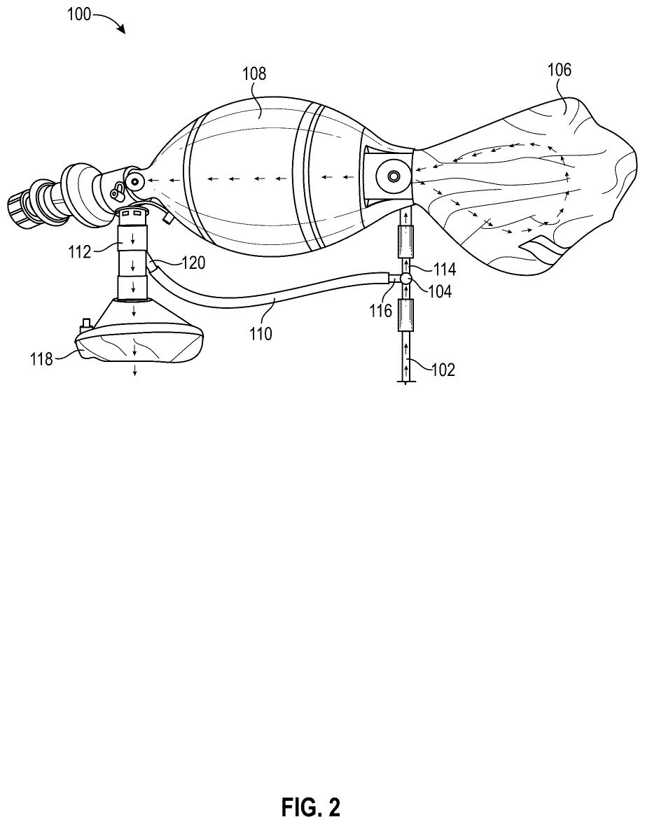

Referring to FIG. 2, once the patient is sedated, the switching valve 104 may be actuated to a second position, closing the outlet tube port 116 and diverting oxygen through the reservoir port 114, where oxygen flows into the reservoir bag 106 and the self-inflating bag 108. A clinician may squeeze the self-inflating bag 108 to assist the breathing of non-spontaneously breathing patient. The oxygen is forced out through the patient port 112 to the patient (such as by using a mask 118 or advanced airways, such as endotracheal tubes). In some embodiments, although not required, an oxygen tube check valve 120 may prevent oxygen from entering the oxygen outlet tube 110 from the patient port 112 when a clinician actuates the self-inflating bag 108.

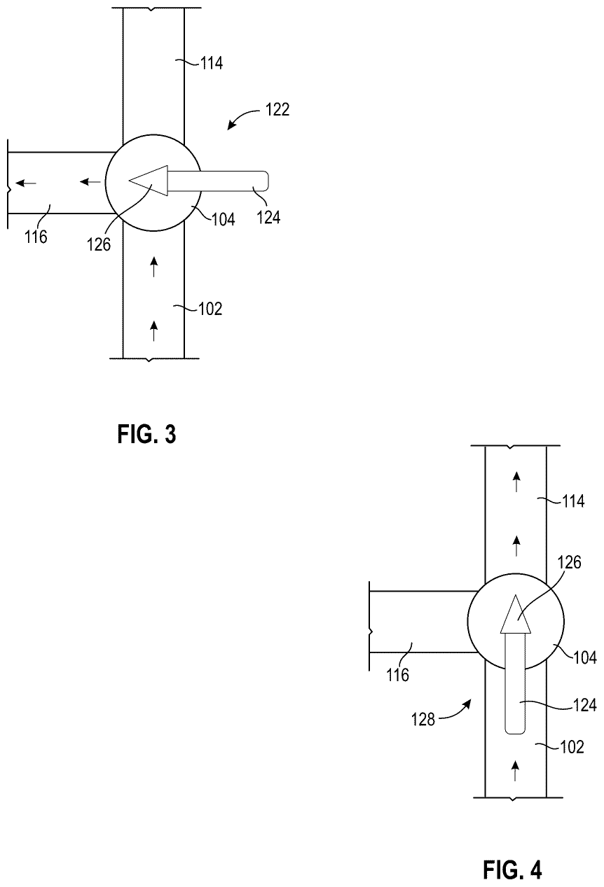

FIGS. 3-4 illustrate, in some embodiments, the switching valve 104 in greater detail. For example, FIG. 3 illustrates the switching valve 104 in a first position 122, wherein oxygen flows from the oxygen inlet 102 to the outlet tube port 116, which is coupled to oxygen outlet tube 110 (not visible in this Fig.). As shown, the switching valve 104 comprises an arm 124 that allows the clinician to actuate the switching valve 104 by pivoting the arm 124. The arm 124 may comprise a head 126 configured to indicate the direction of the flow (flow shown by arrows). It will be understood that while an arm 124 is shown and described, such a configuration is not required, and any mechanism known in the art of valves may be used without departing herefrom.

Referring to FIG. 4, the switching valve 104 is shown in a second position 128. In this position, oxygen flows from the oxygen inlet 102 to the reservoir port 114 and to the reservoir bag 106 and the self-inflating bag 108 (neither of which is visible in this Fig.).

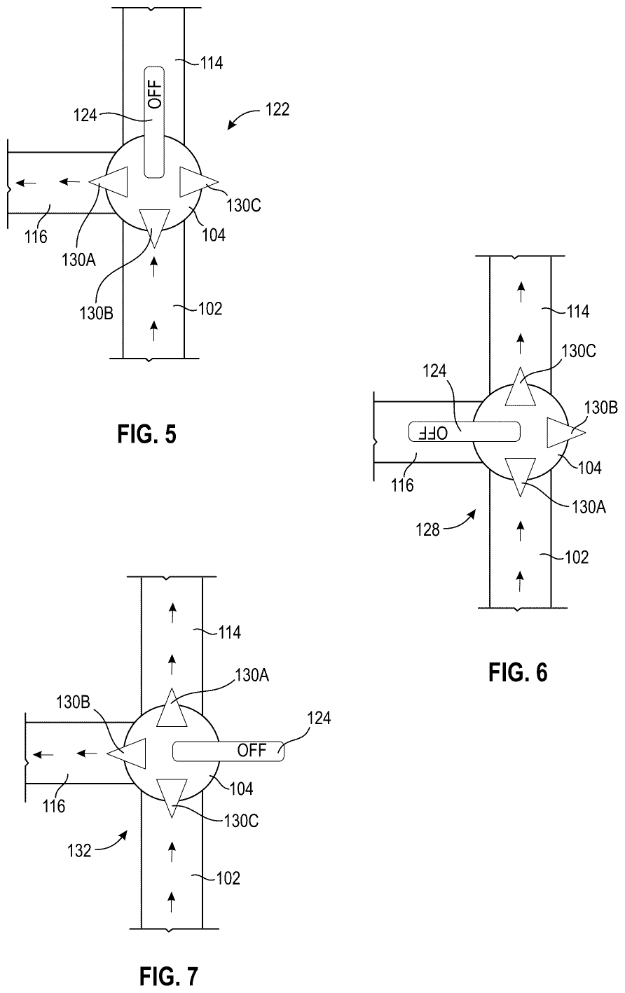

In some embodiments, referring to FIGS. 5-7, the switching valve 104 may comprise three openings (corresponding to indicators 130A-C), allowing the flow of oxygen through one or both of the outlet tube port 116 and reservoir port 114. For example, referring to FIG. 5, the switching valve 104 may be in a first position 122 wherein the reservoir port 114 is closed (the arm 124 operating as the OFF position) and the outlet tube port 116 is open, thereby diverting oxygen (flow of oxygen illustrated by arrows) into the outlet tube port 116 and to the oxygen outlet tube 110 (not shown in this Fig.) and to the patient port 112 (not shown in this Fig.), where it is provided to a spontaneously breathing patient. In other words, oxygen enters through opening 130B and flows through opening 130A into outlet tube port 116. Opening 130C is closed by the valve wall or inlet/outlet wall.

Referring to FIG. 6, the switching valve 104 is in a second position 128, wherein oxygen flows from the oxygen inlet 102 via opening 130A to the reservoir port 114 via opening 130C and to the reservoir bag 106 and the self-inflating bag 108 (neither of which is visible in this Fig.). As shown, the arm 124 is aligned with the oxygen outlet tube port 116, effectively prohibiting oxygen flow into the oxygen outlet tube port 116. Likewise, opening 130B is closed by the valve wall or inlet/outlet wall.

Referring to FIG. 7, the switching valve 104 is in a third position 132, wherein the arm 124 is positioned so as to not close either port 114, 116. In other words, oxygen enters through opening 130C and may simultaneously flow into both the reservoir port 114 via opening 130A and the oxygen outlet tube port 116 via the opening 130B, as indicated by the arrows.

As understood, by simply actuating the switching valve 104, a clinician need not leave the patient's side to switch between oxygen delivery devices, resulting in greater patient care and less potential injury to a patient. In some embodiments, it will be appreciated that the switching valve 104 may be an aftermarket accessory adapted to couple to the inlet/reservoir port 114 of various self-inflating bags in the prior art. For example, the switching valve 104 and oxygen outlet tube 110 may be coupled to a self-inflating bag mask in the prior art, retrofitting current masks in the art and keeping costs low. However, in some embodiments, it will be appreciated that the switching valve 104 may be incorporated or integrated into the oxygen delivery device 100, such as by a manufacturer.

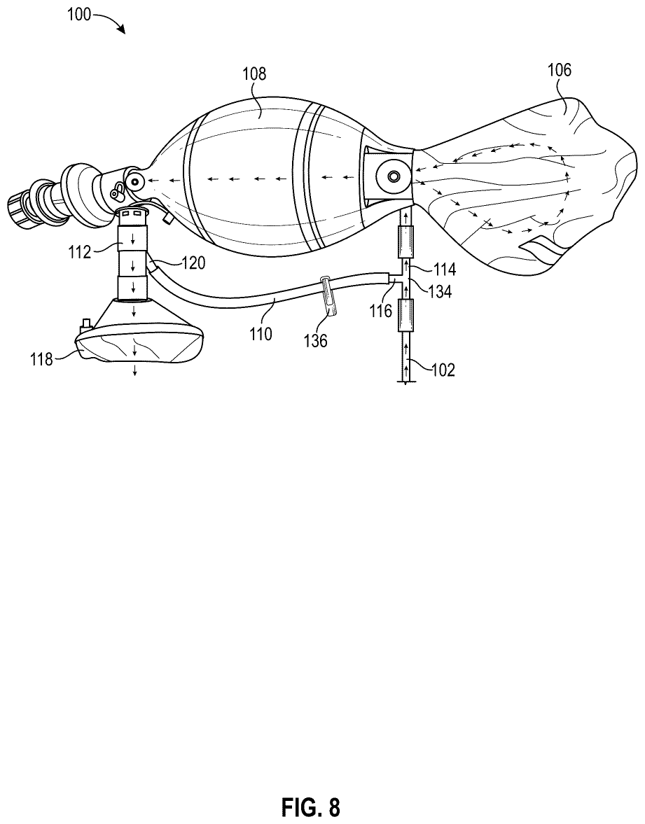

While discussed as a switching valve 104 above, it will be appreciated that, in some embodiments, as shown in FIG. 8, it may simply be a three-way port 134 allowing oxygen to flow simultaneously into the self-inflating bag 108 and reservoir bag 106 as well as the oxygen outlet tube 110. To ensure that the reservoir bag 106 properly inflates and that the self-inflating bag 108 is fully-functional, a clamp 136 may be placed at any location along the length of the oxygen outlet tube 110, thereby blocking oxygen from flowing past the clamp 136 and allowing pressure to properly build in the reservoir bag 106 and self-inflating bag 108.

In this embodiment, the clamp 136 would remain open while the patient is spontaneously breathing, allowing oxygen to flow directly to the patient through the oxygen outlet tube 110 and through the mask 118. Once the patient ceases to breathe spontaneously, the clinician may actuate the clamp 136 to close the clamp 136, effectively sealing the oxygen outlet tube 110 and prohibiting the flow of oxygen past the clamp 136, thereby forcing all the oxygen to flow into the reservoir bag 106 and self-inflating bag 108, wherein the self-inflating bag 108 may then be actuated by the clinician to provide oxygen to the patient, mimicking natural breathing. As prior embodiments, the oxygen outlet tube 110 allows a clinician to maintain maximum focus on the patient and easily switch between oxygen supplies without diverting attention from the patient. While a switching valve 104 and a clamp 136 were used as examples herein (both a type of directional flow device), other directional flow devices known in the art may be used without departing herefrom.

Accordingly, it will be appreciated from the foregoing that the oxygen delivery device 100 disclosed herein solves the need for an oxygen mask that is capable of delivering free-flowing oxygen to a spontaneously breathing patient, while further providing a self-inflating resuscitation bag once the patient is not spontaneously breathing.

It will be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. An oxygen delivery device, comprising:
an oxygen inlet;
a switching valve coupled to the oxygen inlet;
an oxygen outlet tube coupled on a first end to the switching valve, the oxygen outlet tube coupled on a second end to a mask;
a reservoir port coupled to the switching valve;
a reservoir bag coupled to the reservoir port; and
a self-inflating bag coupled to the reservoir port and reservoir bag, the self inflating bag coupled to the mask.

2. The oxygen delivery device of claim 1, wherein when the switching valve is in a first position, oxygen flows through the oxygen inlet, through the switching valve, through the oxygen outlet tube, and to the mask.

3. The oxygen delivery device of claim 1, wherein when the switching valve is in a second position, oxygen flows through the oxygen inlet, through the switching valve, through the reservoir port, through the reservoir bag and the self-inflating bag, and to the mask.

4. The oxygen delivery device of claim 1, wherein when the switching valve is in a third position, oxygen flows simultaneously:
a. through the oxygen inlet, through the switching valve, through the reservoir port, through the reservoir bag and the self-inflating bag, and to the mask, and
b. through the oxygen inlet, through the switching valve, through the oxygen outlet tube, and to the mask.

5. The oxygen delivery device of claim 1, further comprising an oxygen tube check valve interposed between the mask and the switching valve.

6. An oxygen delivery device, comprising:
an oxygen inlet;
a three-way port coupled to the oxygen inlet;
an oxygen outlet tube coupled on a first end to the three-way port, the oxygen outlet tube coupled on a second end to a mask;
a clamp positioned on the oxygen outlet tube;
a reservoir port coupled to the three-way port;
a reservoir bag coupled to the reservoir port; and
a self-inflating bag coupled to the reservoir port and reservoir bag, the self inflating bag coupled to the mask.

7. The oxygen delivery device of claim 6, wherein when the clamp is in an open position, oxygen flows through the oxygen inlet, through the three-way port, through the oxygen outlet tube, and to the mask.

8. The oxygen delivery device of claim 6, wherein when the clamp is in a closed position, oxygen flows through the oxygen inlet, through the three-way port, through the reservoir port, through the reservoir bag and the self-inflating bag, and to the mask, the clamp prohibiting oxygen from flowing through the oxygen outlet tube to the mask.

9. An oxygen delivery device, comprising:
an oxygen inlet;
an oxygen outlet tube coupled on a first end to the oxygen inlet, the oxygen outlet tube coupled on a second end to a mask;
a reservoir port coupled to the oxygen inlet;
a reservoir bag coupled to the reservoir port;
a self-inflating bag coupled to the reservoir port and reservoir bag, the self inflating bag coupled to the mask; and
a directional flow device configured to direct a flow of oxygen through the oxygen outlet tube or to prohibit the flow of oxygen through the oxygen outlet tube.

10. The oxygen delivery device of claim 9, wherein the directional flow device is a switching valve.

11. The oxygen delivery device of claim 10, wherein when the switching valve is in a first position, oxygen flows through the oxygen inlet, through the switching valve, through the oxygen outlet tube, and to the mask.

12. The oxygen delivery device of claim 10, wherein when the switching valve is in a second position, oxygen flows through the oxygen inlet, through the switching valve, through the reservoir port, through the reservoir bag and the self-inflating bag, and to the mask.

13. The oxygen delivery device of claim 10, wherein when the switching valve is in a third position, oxygen flows simultaneously:
a. through the oxygen inlet, through the switching valve, through the reservoir port, through the reservoir bag and the self-inflating bag, and to the mask, and
b. through the oxygen inlet, through the switching valve, through the oxygen outlet tube, and to the mask.

14. The oxygen delivery device of claim 9, wherein the directional flow device is a clamp positioned on the oxygen outlet tube.

15. The oxygen delivery device of claim 14, wherein when the clamp is in an open position, oxygen flows through the oxygen inlet, through the oxygen outlet tube, and to the mask.

16. The oxygen delivery device of claim 14, wherein when the clamp is in a closed position, oxygen flows through the oxygen inlet, through the reservoir port, through the reservoir bag and the self-inflating bag, and to the mask, the clamp prohibiting oxygen from flowing through the oxygen outlet tube to the mask.

17. The oxygen delivery device of claim 14, further comprising an oxygen tube check valve interposed between the mask and the clamp.
